(19) United States
(12) Patent Application Publication
(10) Pub. No.: US 2016/0168224 A1
(43) Pub. Date: Jun. 16, 2016

(54) PROCESS FOR PREPARING AMPHIPHILIC PEPTIDE DERIVATIVES

(71) Applicant: UNIVERSIDADE FEDERAL DE MINAS GERAIS - UFMG, Belo Horizonte (BR)

(72) Inventors: Janaína Félix BRAGA, Belo Horizonte (BR); Frederic Jean Georges FREZARD, Belo Horizonte (BR); Robson Augusto SOUZA dos SANTOS, Belo Horizonte (BR); Diógenes SOUSA NETO, Belo Horizonte (BR); Mariana Flávia de OLIVEIRA, Belo Horizonte (BR)

(21) Appl. No.: 14/904,430

(22) PCT Filed: Jun. 11, 2014
(86) PCT No.: PCT/IB2014/062135

§ 371 Date: Jan. 11, 2016

Related U.S. Application Data
(60) Provisional application No. 61/833,909, filed on Jun. 11, 2013.

(51) Int. Cl. C07K 14/575

(52) U.S. Cl. CPC C07K 14/575; A61K 38/00

(57) ABSTRACT

The present invention relates to novel derivatives of hydrophilic bioactive peptides which have been modified by introducing a lipophilic substituent through esterification of the free carboxylic group(s) of the peptide with an aliphatic alcohol. The present invention also refers to a simple, fast and effective process for the preparation of amphiphilic derivatives of peptides. It also refers to pharmaceutical compositions for the controlled release and delivery of these pharmacologically active peptides.

### BACKGROUND OF THE INVENTION

Natural or synthetic peptides have shown promise as pharmaceutics with the potential to treat a wide variety of diseases. Peptides are usually selective and efficacious, therefore need only be present in low concentrations to act on their targets. The metabolism of peptides is superior to other small molecules due to the limited possibility for accumulation and result in relatively non-toxic amino acid, peptide metabolites. These properties contribute towards the overall low toxicity of peptides, with a limited risk of adverse interactions.

On the other hand, the potential of peptides as drugs is often overshadowed by their inability to reach their targets in an active form in vivo. The delivery of active peptides is challenging due to inadequate absorption through the mucosa or the skin and rapid breakdown by proteolytic enzymes. Furthermore, most peptides are quickly degraded in serum and exhibit rapid clearance in vivo. Many strategies have been employed in an attempt to overcome these disadvantages, including chemical modifications of the peptide and delivery strategies based on physical or pharmaceutical technologies (Benson H A E, Namjoshi S. Proteins and Peptides: Strategies for Delivery to and Across the Skin. J Pharm Sci 97:3591-3610, 2008; Renukuntla J, Vadlapudi A D, Patel A, Boddu S H S, Mitra A K. Approaches for enhancing oral bioavailability of peptides and proteins. Int J Pharm 447:75-93, 2013; Singh N, Kalluri H, Herwadkar A, Badkar A, Banga A K. Transcending the Skin Barrier to Deliver Peptides and Proteins Using Active Technologies. Crit Rev Therap Drug Carrier Syst, 29:265-298, 2012).

Chemical modifications of peptides include lipidation, glycosylation, cyclization, backbone modification, unnatural or D-amino acid conjugation and PEGylation (Goodwin D, Simerska P, Toth I. Peptides As Therapeutics with Enhanced Bioactivity. Curr Med Chem 19:4451-4461, 2012). Among these, lipidation has proven to be one of the most robust strategies for the generation of new therapeutic peptide leads as discussed in details in a recent review paper (Zhang L, Bulaj G. Converting Peptides into Drug Leads by Lipidation. Current Medicinal Chemistry, 2012, 19, 1602-1618; U.S. Pat. No. 8,518,876 B2).

Lipidation can dramatically change peptides' physicochemical and pharmacological properties. Additionally, lipidation may increase peptides plasma stability and decrease peptides kidney clearance. When the chemical bond between lipid moiety and the parental peptide is cleavable by specific enzyme, the peptide derivative can be considered as a pro-drug. The expected benefits of lipidation are improved bioavailabiilty by oral, mucosal and transdermal routes. Slow rate of activation of the peptide prodrug and/or its slow renal clearance can also result in long-lasting action of the peptide. As another advantage, lipidation allows incorporation of the peptide derivative into the hydrophobic phase of a carrier system, such as micelles, lipid-based nanoparticles or polymeric carrier, with further improvement of its stability and action and the possibilty of targeting to specific body sites.

Several different synthetic processes have been used for the lipidation of peptides, however, most of them relates to the conjugation of fatty acid and are complex processes which involve several reaction steps (Zhang L, Bulaj G. Converting Peptides into Drug Leads by Lipidation. Current Medicinal Chemistry, 2012, 19, 1602-1618).

The heptapeptide Angiotensin-(1-7) (Ang-(1-7) or Asp¹-Arg²-Val³-Tyr⁴-Ile⁵-His⁶-Pro⁷) is an important component of the renin-angiotensin system (RAS) with pleiotropic actions, including vasodilator, antiproliferative, antifibrotic, antiarrhythmic, antihypertrophic and antithrombotic effects, which are mediated mainly by the G protein-coupled receptor Mas (Santos R A S. Angiotensin-(1-7). Hypertension 63:1138-47, 2014). In the RAS, Angiotensin-II peptide often promotes opposite actions to that of Ang-(1-7), through binding to the AT1-receptor.

Because of its beneficial biological actions, the peptide Ang-(1-7) serves as a basis for the development of new pharmacotherapeutic drugs for treatment of hypertension, heart failure, cardiac hypertrophy, diabetic complications, metabolic syndrome, artherosclerosis, cancer, muscular dystrophy, glaucoma, erectile dysfunction and alopecia (Santos R A S. Angiotensin-(1-7). Hypertension 63:1138-47, 2014; WO 01/98325).

Several natural and synthetic analogs of Ang-(1-7) were studied, with the aim of identifying more effective peptide drug (Santos R A S. Angiotensin-(1-7). Hypertension 63:1138-47, 2014; Lautner et al. Discovery and characterization of alamandine: a novel component of the renin-5 angiotensin system. Circulation Research 112:1104-1111, 2013; WO 01/98325). Among these, the endogenous peptide alamandine, with substitution of Asp for Ala at the N-terminal position of Ang-(1-7), showed vasodilating action similar to Ang-(1-7), through binding to a receptor different from that of Ang-(1-7). This also suggests possible sinergistic effects of Ang-(1-7) and alamandine.

As endogenous peptides, Ang-(1-7) and Alamandine have great advantage of exhibiting a low toxicity profile. Mordwinkin et al. (Mordwinkin et al. Toxicological and Toxicokinetic Analysis of Angiotensin (1-7) in Two Species. J Pharm Sci 101:373-80, 2012) showed that subcutaneous administration of Ang-(1-7) at 10 mg/(kg day) for 28 days did not lead to any detectable toxicities in either rats or dogs. No toxicicity was reported in human subjects submitted to anticancer chemotherapy, after receiving the peptide subcutaneously at 300 μg/kg (Pham et al. Pharmacodynamic stimulation of thrombogenesis by angiotensin-(1-7) in recurrent ovarian cancer patients receiving gemcitabine and platinum-based chemotherapy. Cancer Chemother Pharmacol 71:965-72, 2013). On the other hand, the rapid in vivo metabolism of the peptide through inactivation by proteolytic enzymes results in a very short plasma half-life, typically about 10 min in rodents and 30 min in humans (Yamada et al. Converting enzyme determines plasma clearance of angiotensin-(1-7). Hypertension 32:496-502, 1998; Rodgers et al. Expression of intracellular filament, collagen, and collagenase genes in diabetic and normal skin after injury. Wound Repair Regen 14:298-305, 2006), and short biological actions, limiting its therapeutic potential. Furthermore, the high molecular weight and hydrophilic and peptidergic character of Ang-(1-7) are physicochemical factors that limit its absorption across biological barriers, such as the skin or gastrointestinal tract.

Biologically active lipidated antagonists of angiotensin II have been obtained through solid phase peptide synthesis using the Boc strategy (Maletinska et al. Lipid Masking and Reactivation of Angiotensin Analogues. Helv Chim Acta 79, 2023-34, 1996; Maletinska et al. Angiotensin Analogues Palmitoylated in Positions 1 and 4. J. Med. Chem. 40:3271-79, 1997).

### SUMMARY OF INVENTION

The present invention relates to novel derivatives of hydrophilic bioactive peptides and to methods of making and using them. Thus, in its broadest aspect, it relates to pharmacologically active peptides which have been modified by introducing a lipophilic substituent, through esterification of the free carboxylic group(s) of the peptide. Esterification of the free carboxylic group(s) of the peptide is achieved with a high yield, through a rapid single-step reaction of the peptide with an aliphatic alcohol, in a water-in-alcohol emulsion.

In one preferred embodiment of the present invention, the aliphatic alcohol is liquid at temperature below 75° C.

In another preferred embodiment of the present invention, the aliphatic alcohol is 1-octanol.

In another preferred embodiment of the present invention, the bioactive peptide contains 2 to 100 amino acids.

In another preferred embodiment of the present invention, the bioactive peptide contains at least one Asp or Glu.

In another preferred embodiment of the present invention, the bioactive peptide is Ang-(1-7) or an analogue.

In another preferred embodiment, the present invention relates to the use of the peptide derivative(s) of the invention as therapeutic agents.

In another preferred embodiment, the present invention relates to therapeutic agents, pharmaceutical compositions or drug delivery devices containing the peptide derivative(s) of the invention.

In another preferred embodiment, the present invention relates to a pharmaceutical composition or a drug delivery device for treatment by topical, oral, nasal, pulmonary, intravenous, transdermal or subcutaneous route of cardiometabolic diseases, including hypertension, metabolic syndrome, cardiac hypertrophy, stroke, muscular dystrophy, glaucoma, erectil dysfunction or alopecia, comprising a therapeutically effective amount of Ang-(1-7) derivative(s) according to the invention together with a pharmaceutically acceptable carrier or excipient.

In another preferred embodiment, the pharmaceutically acceptable carrier relates to a drug carrier or delivery device for the controlled release of the peptide derivative(s) including, but not limited to, cyclodextrins, polymers, mucoadhesive polymer, lipid vesicles, polymersomes, solid lipidic nanoparticles, polymeric micro and nanoparticles, micro- and nanocapsules, micro- and nanoemulsions, dendrimers, micelles, polymeric micelles, inorganic nanoparticles, carbon nanoparticles, transdermal patches, implantable polymeric matrix or associations of these systems.

In another preferred embodiment, the present invention relates to a method of treating cardiometabolic diseases, including hypertension, syndrome metabolic, cardiac hypertrophy, stroke, muscular dystrophy, glaucoma, erectil dysfunction or alopecia in a patient in need of such a treatment comprising administering to the patient a therapeutically effective amount of Ang-(1-7) derivative(s) according to the invention together with a pharmaceutically acceptable carrier or excipient.

### BRIEF DESCRIPTION OF THE DRAWINGS

FIG. 1. FTIR spectra of the Ang-(1-7) A) before and B) after reaction with 1-octanol.

FIG. 2. ESIMS (positive ion mode) for the Ang-(1-7) peptide A) before and B) after esterification with 1-octanol.

FIG. 3. HPLC chromatogram of Ang-(1-7) A) before and B) after esterification reaction with 1-octanol. The Arabic numbers refer to the non-sterified (1), monosterified (2 and 3) and diesterified peptides (4).

FIG. 4. Changes in mean arterial blood pressure (MAP) in spontaneously hypertensive rats as a function of time, after topical application of amphiphilic Ang-(1-7) derivatives (20 μl at 1 mg/ml). Data are shown as means±SEMs (n=3). Formulation was applied at t=0.

### DETAILED DESCRIPTION OF THE INVENTION

Specifically, this invention refers to some new amphiphilic angiotensin peptides and their pharmaceutical compositions which are effective when administered by topical, oral, nasal, pulmonary, intravenous, transdermal, subcutaneous routes. These amphiphilic angiotensin peptide derivatives and their pharmaceutical compositions should find application in the treatment of several cardiometabolic diseases, such as hypertension, syndrome metabolic, cardiac hypertrophy, stroke, muscular dystrophy, glaucoma, erectil dysfunction. These derivatives could be also used in cosmetic formulations for treating or preventing allopecie and as anti-apoptotic agents.

The process claimed in the present invention refers to a reaction of esterification between the peptide and an aliphatic alcohol, in a very simple and effective manner using a water-in-oil emulsion system, so as to obtain amphiphilic peptide derivative(s). This process can be applied to any aliphatic alcohol or derivative with melting point below 75° C. No such process has been described previously in the state of the technique.

According to a preferred embodiment of the invention, the process comprises steps of:

i) Acidifying an aliphatic alcohol having melting point lower than 75° C. with a strong acid;

ii) Mixing the acidified aliphatic alcohol with a peptide;

iii) Heating the mixture at a temperature in the range of about 25 to 75° C.;

and iv) Extracting the resulting amphiphilic peptide ester derivative(s);

Preferred process comprises adding HCI 36.5% to the alcohol in the liquid state, typically 10-to-2000 μL of HCI 36.5% to 1-to-1000 mL of aliphatic alcohol. Acidified aliphatic alcohol is then added to peptide powder, typically 0.5-to-1000 mL of acidified aliphatic alcohol to 5-to-5000 mg of peptide. The mixture is heated, typically between 25-to-75° C., and the dispersion is maintained under agitation, for instance using a magnetic bar (flea). After 1-to-24 h of reaction, the non-reacted peptide can be removed, for instance by filtration or centrifugation. The extration of amphiphilic peptide derivatives from the aliphatic alcohol reaction medium can then be performed, for instance by adding an excess of cyclohexane, allowing for the precipitation of the reaction products. To remove the residue of aliphatic alcohol, the product can be washed with cyclohexane. The resulting product can be dispersed in water and the suspension freeze-dried to remove any trace of solvent.

As shown in Example 1, this process was successfully applied to the synthesis of an amphiphilic Ang-(1-7) derivatives from 1-octanol. The main derivative formed was found to be the peptide diesterified at the C-terminal and Asp carboxyl groups. Peptide derivatives monoesterified, at either the C-terminal or Asp carboxyl group, were also identified in the product of the reaction.

Interestingly, the reaction of Alamandine with 1-octanol did not show the formation of ester derivative by ESI-MS. Since Alamandine differs from Ang-(1-7) only by a substitution of Asp for Ala at the N-terminal position, one can infer that the peptide should contain at least one amino acid with a carboxylic group (Asp or Glu) for the esterification reaction to occur.

The present invention is further illustrated by the following examples which, however, are not to be construed as limiting the scope of protection.

### EXAMPLE 1

### Synthesis and Characterization of Amphiphilic Angiotensin-(1-7) Derivative(s) from Esterification with 1-Octanol

1-octanol (5 mL) was acidified with hydrochloric acid 36.5% (200 μL) in a round bottom glass tube. Under strong agitation, 25 mg of peptide was dissolved. The tube was sealed and peptide was left to react for approximately 1 h at 60° C. The organic solution was transferred to a falcon tube and 20 mL of cyclohexane was added. The tube was shaken on a vortex mixer and centrifuged at 10,000×g for 20 min at 10° C. The pellet was washed twice with 10 mL of cyclohexane under the same centrifugation conditions. The residual solvent was removed by placing the sample in a glass vacuum desiccator for 12 h. The tube was sealed in an argon atmosphere and stored at −20° C. until use. The reaction yield was higher than 70%. This yield was estimated based on intrinsic fluorescence of peptide derivative in methanol, using the Ang-(1-7) as standard sample.

The product was characterized by Fourier Transform Infrared (FTIR), Electrospray Ionization mass spectrometry (ESI-MS) in the positive mode and High performance liquid chromotography (HPLC).

FTIR spectra were acquired on a Thermo Scientific FTIR spectrometer equipped with a broad-band mercury cadmium telluride detector and an attenuated total reflectance (ATR) accessory. The spectral parameters used during the kinetic experiments were: speed, 20 kHz; filter, 5 kHz; UDR 2; resolution, 1 cm⁻¹; and a triangular apodization function. These measurements were carried out with a time resolution of 400 s and length of run equal to 400 min. At this configuration, each spectrum is an average of 249 scans. A small aliquot (20 μL) of peptide in a mixture of chloroform:methanol (1:3, v/v) was placed on a germanium ATR crystal and, after solvent evaporation, the infrared spectra was recorded.

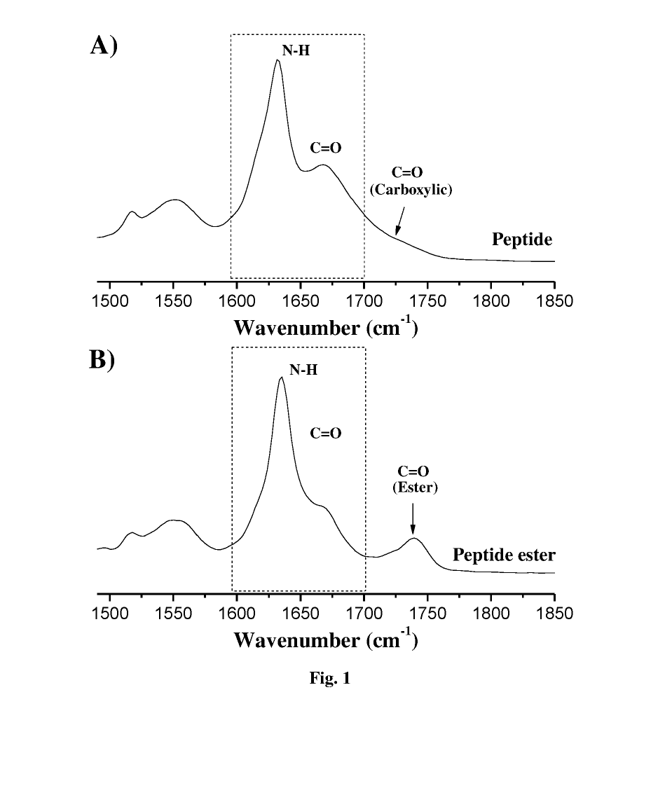

FIG. 1 shows the FTIR spectra for the Ang-(1-7) before A) and after B) reaction with the fatty alcohol, within a frequency range 1500-1850 cm⁻¹. As demonstrated in FIGS. 1A and 1B, the peptide amide I band lineshape (see dashed rectangle) did not change upon esterification, which is an indicative that peptide backbone is preserved after reaction. The main spectral changes refer to the C═O stretching frequency from carboxylic groups. This chemical group is found at the C-terminus of peptide, as well as in the aspartic acid at the N-terminus. Before reaction, the C═O stretching band is centered around 1725 cm⁻¹, which is a typical frequency value for carboxylic groups. This band is markedly decreased in the product of reaction and another band with maxima at 1740 cm⁻¹ is observed in the FTIR spectra (see FIG. 1B). This band is associated to the C═O stretching of the ester group and indicates that peptide undergoes esterification with 1-octanol.

ESI-MS spectra were acquired on a Shimadzu high-performance liquid chromatography coupled to mass spectrometer (LCMS-IT-TOF), using capillary cone heated at 200° C., 1.63 kV spray voltage with nitrogen and interface voltage at −3.5 kV. The samples were solubilized in a mixture of chloroform:methanol (1:3, v/v) and their mass spectra (positive ion mode) were recorded in a range from m/z 50-1000.

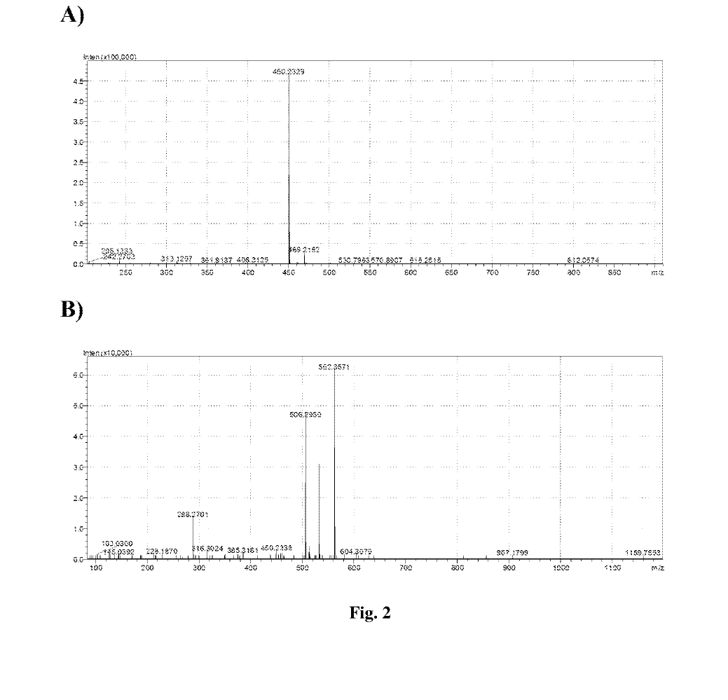

FIG. 2 shows the ESIMS (positive ion mode) for Ang-(1-7) A) before and B) after reaction with this fatty alcohol. Spectral analysis revealed that isotopes are separated by ½ mass unit, indicating that the ionic species are doubly charged. In FIG. 2A, a main double-protonated species with mass of 900.5 Da was observed, corresponding to the Ang-(1-7) peptide. After reaction (see FIG. 2B), two double-charged species with mass of 1012.6 Da and 1124.7 Da were detected, that can be assigned to the mono- and di-esterified forms of Ang-(1-7), respectively. Indeed, these mass values are in agreement with a molecular weight increase corresponding that of 1-octanol minus that of water (released from the esterification reaction). A third double-protonated species with mass of 1065 Da can be assigned to the mono-esterification of secondary species (mass at 938 Da) detected in the Ang-(1-7) spectrum.

A Shimadzu HPLC system equipped with UV-VIS detector was used to further investigate the chemical species in the reaction product. Reverse-phase HPLC was performed using a Vydac C18 column (4.6 mm×250 mm) with a particle size of 5 μm. A 10 μL aliquot of the sample (parent peptide or reaction product) in acetonitrile was injected onto the HPLC column. The mobile phase consisted of solvent A (0.13% heptafluorobutyric acid and 99.87% water) and solvent B (0.13% heptafluorobutyric acid, 80% acetonitrile and 19.87% water). The species were eluted using a gradient elution program of 20% to 100% B in 80 min, a 20-min hold at 100% B, followed by a return to 20% B for a 10-min equilibration. The flow rate was 0.5 mL/min.

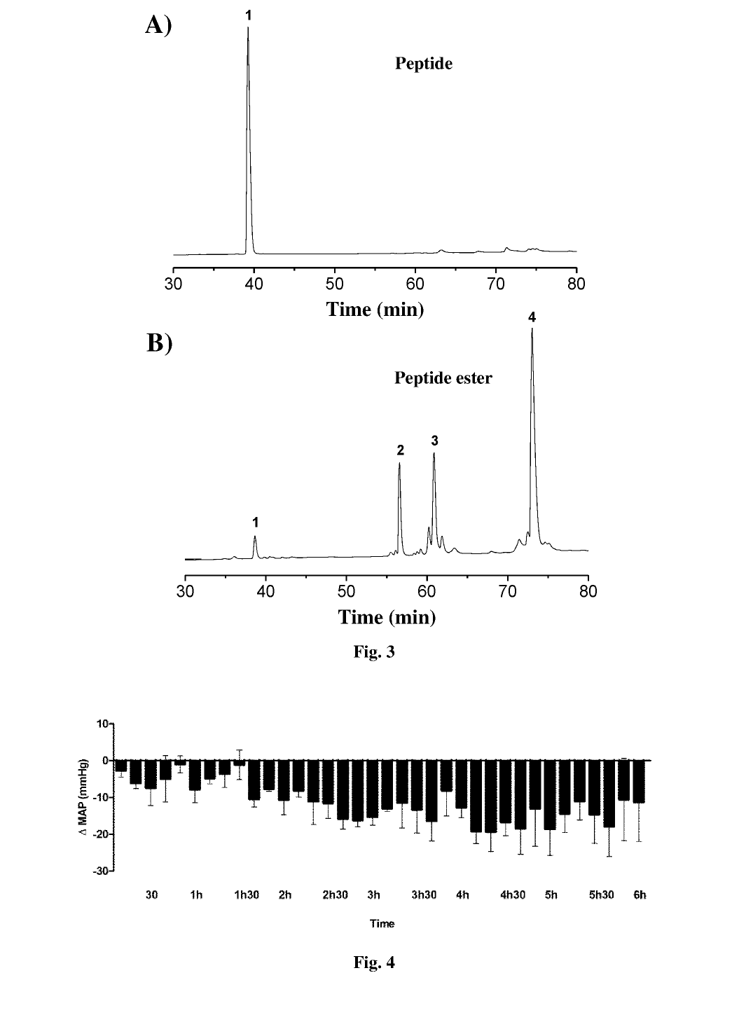

FIG. 3 shows the HPLC chromatograms obtained for this peptide before A) and after B) esterification. In FIG. 3A, peak 1 corresponds to the Ang-(1-7), whereas the additional peaks present in FIG. 2B are associated to the peptide esters. Peaks 2 and 3 can be assigned to the peptide monoester derivatives that are formed by the 1-octanol bound either at the C-terminus of peptide or at the aspartic acid carboxyl group. Since their affinity to the column stationary phase is different, both peptide monoesters could be separated. The predominant species, which gives the more intense Peak 4, can be attributed to the di-ester peptide derivative. This molecule is more hydrophobic than the mono-esterified forms and, therefore, its corresponding peak is observed at a higher elution time in the chromatogram.

### EXAMPLE 2

### Biological Action of Amphiphilic Peptide Derivatives After Topical Application in Spontaneously Hypertensive Rats

To evaluate the ability of the amphiphilic peptide derivatives to exert systemic biological action after topical application, spontaneously hypertensive rats (SP-SHR) and Ang-(1-7) derivatives, as prepared in Example 1 were used.

SP-SHR rats (4-5 months-old with about 270 g body weight) were instrumented for acute blood pressure measurements using tribromoethanol anesthesia. Just after the arterial catheter implantation, the interscapular region was shaved for topical application of the formulation. Blood pressure (BP) was recorded 24 h after surgery (base line BP=173.0±6.2). BP was recorded in unanesthesized animals under resting conditions for 1 h. After that, a solution of Ang-(1-7) derivatives was applied topically (20 μl at 1 mg/ml) and BP was continuously recorded for 6 h.

As illustrated in FIG. 4, application of Ang-(1-7) ester derivatives promoted a marked decrease of BP up to 25 mmHg, starting at 90 min and lasting for up to 6 h.

The topical application of the non-modified Ang-(1-7) peptide did not cause significant change in BP. This example establishes the potential of amphiphilic Ang-(1-7) derivatives in topical formulations such as transdermal patches, for promoting a long-lasting systemic biological action. It also establishes their potential for the sustained control of BP.

1. A process for preparation of amphiphilic derivatives of a peptide comprising an esterification reaction of the peptide with an aliphatic alcohol having melting point lower than 75° C.

2. The process of claim 1 comprising the steps of:
(a) acidifying the aliphatic alcohol having melting point lower than 75° C. with a strong acid;
(b) mixing the acidified aliphatic alcohol and a peptide;
(c) heating the mixture at a temperature in the range of about 25° C. to about 70° C.; and
(d) extracting the resulting amphiphilic peptide derivatives.

3. The process of claim 1, wherein the peptide contains 2 to 100 amino acids.

4. The process of claim 1, wherein the peptide contains at least one Asp or Glu.

5. The process of claim 1, wherein the peptide is Angiotensin-(1-7).

6. The process of claim 1, wherein the aliphatic alcohol is 1-octanol.

7. A pharmaceutical composition or drug delivery device comprising an amphiphilic derivative of a peptide or a mixture of amphiphilic derivatives of a peptide, obtained by the process according to claim 1.

8. The pharmaceutical composition or drug delivery device according to claim 7, which further comprises a controlled release and delivery system such as cyclodextrin(s), polymer(s), mucoadhesive polymer(s), lipid vesicles, polymersomes, solid lipidic nanoparticles, polymeric micro- or: nanoparticles, micro- or nanocapsules, micro- or nanoemulsions, dendrimers, micelles, polymeric micelles, inorganic nanoparticles, carbon nanoparticles, transdermal patches, implantable polymeric matrix, or an association of these systems.

9. The pharmaceutical composition or drug delivery device according to claim 7, wherein the mixture of amphiphilic peptide derivatives are ester derivatives of Ang-(1-7) with an aliphatic alcohol.

10. The pharmaceutical composition or drug delivery device according to claim 9, wherein the aliphatic alcohol is 1-octanol.

11. The pharmaceutical composition or drug delivery device according to claim 7, wherein the amphiphilic peptide derivative is Ang-(1-7) esterified with an aliphatic alcohol at the carboxyl group of Asp.

12. The pharmaceutical composition or drug delivery device according to claim 11, wherein the aliphatic alcohol is 1-octanol.

13. The pharmaceutical composition or drug delivery device according to claim 7, wherein the amphiphilic peptide derivative is Ang-(1-7) esterified with an aliphatic alcohol at the C-terminal carboxyl group.

14. The pharmaceutical composition or drug delivery device according to claim 13, wherein the aliphatic alcohol is 1-octanol.

15. The pharmaceutical composition or drug delivery device according to claim 7, wherein the amphiphilic peptide derivative is Ang-(1-7) diesterified with an aliphatic alcohol at both C-terminal and Asp carboxyl groups.

16. The pharmaceutical composition or drug delivery device according to claim 15, wherein the aliphatic alcohol is 1-octanol.

17. A pharmaceutical composition or drug delivery device for treatment by topical, oral, nasal, pulmonary, intravenous, transdermal, or subcutaneous route of cardiometabolic diseases, including hypertension, metabolic syndrome, cardiac hypertrophy, stroke, muscular dystrophy, glaucoma, erectile dysfunction, or alopecia; comprising a therapeutically effective amount of Ang-(1-7) derivative(s) obtained by the process according to claim 1, together with a pharmaceutically acceptable carrier or excipient.

18. A method of treating cardiometabolic diseases, including hypertension, syndrome metabolic, cardiac hypertrophy, stroke, muscular dystrophy, glaucoma, erectile dysfunction, or alopecia in a patient in need of such a treatment; comprising administering to the patient a therapeutically effective amount of Ang-(1-7) derivative(s) obtained by the process according to claim 1, together with a pharmaceutically acceptable carrier or excipient.
